(19) United States
(12) Patent Application Publication
(10) Pub. No.: US 2007/0185392 A1
(43) Pub. Date: Aug. 9, 2007

(54) Method and device for indicating moisture content of skin

(76) Inventors: Faiz Feisal Sherman, West Chester, OH (US); Vladimir Gartstein, Cincinnati, OH (US); David Burton Moore, Hamilton, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/784,132

(22) Filed: Apr. 5, 2007

Related U.S. Application Data
(62) Division of application No. 11/514,645, filed on Sep. 1, 2006.
(60) Provisional application No. 60/713,768, filed on Sep. 2, 2005.

(51) Int. Cl. A61B 5/00; A61B 5/04

(52) U.S. Cl. 600/306; 600/372; 600/393

(57) ABSTRACT

A device for providing an indication of moisture present in skin of a user includes a device body including a skin contact portion. A sensor circuit is located in the device body. The sensor circuit is configured to produce an output signal having a signal value that corresponds to moisture level in the skin with the skin contact portion in contact with the skin at a skin measurement location. A controller is in communication with the sensor circuit and is configured to determine the signal value of the output signal. The controller stores a first signal value in memory corresponding to a first output signal produced with the skin contact portion in contact with the skin of the user at a first skin measurement location. The controller stores a second signal value in memory corresponding to a second output signal generated with the contact portion in contact with the skin of the user at a second skin measurement location different from the first skin measurement location. The first and second signal values are used to indicate moisture present in the skin of the user at the second skin measurement location.

### CROSS REFERENCE TO RELATED APPLICATIONS

This application is a divisional of U.S. application Ser. No. 11/514,645, filed Sep. 1, 2006, which claims the benefit of U.S. Provisional application Ser. No. 60/713,768, filed on Sep. 2, 2005.

### TECHNICAL FIELD

The present application relates generally to a method and device for indicating moisture content of skin.

### BACKGROUND

Skin is subject to insults by many extrinsic and intrinsic factors. Extrinsic factors include ultraviolet radiation (e.g., from sun exposure), environmental pollution, wind, heat or infrared radiation (IR), low humidity, harsh surfactants,. abrasives, and the like. Intrinsic factors include chronological aging and other biochemical changes from within the skin. Whether extrinsic or intrinsic, these factors result in visible signs of skin aging and environmental damage, such as wrinkling and other forms of roughness (including increased pore size, flaking and skin lines), and other histological changes associated with skin aging or damage. Additionally, the water content of the stratum corneum has a profound influence on the appearance, flexibility, texture, and dryness of the skin, and also on the absorption of drugs and other molecules into and through the skin. The stratum corneum is the outermost layer of the epidermis, and comprises the surface of the skin

Methods of treating the skin generally involve the application of at least one of a variety of appropriate treatments. Such treatments may be selected to provide or to restore certain desired physical or cosmetic characteristics to the skin or scalp. However, unless an appropriate treatment is selected, the desired physical or cosmetic characteristic may not be obtained.

In the case of treating skin, such as the scalp, treatments generally include shampoos, conditioners, colorants, styling compositions, and the like. Manufacturers of these scalp treatments may provide multiple versions of a type or brand of scalp treatment, wherein each of the multiple versions is specifically designed to target a need or demand which is characteristic of a specific consumer segment and which may be based on physical or cosmetic differences of the scalp generally found between respective consumer segments. For example, a single brand shampoo may offer a first version designed to treat flakes and a second version designed to treat dryness, both conditions associated with dandruff.

However, when a consumer is faced with the task of selecting a scalp treatment from among the multiple versions of a scalp care brand, the consumer may unknowingly select a version which is not designed to provide the characteristics desired. by the consumer. In such a case, the consumer may be dissatisfied with the results of the selected version of the scalp care brand. As a result of the consumer's dissatisfaction, the consumer subsequently may refuse to select any of the versions of that same scalp care brand even though another version of that scalp care brand may provide the consumer's desired scalp and/or hair characteristics. The occurrence of such circumstances, in turn, may lead to unnecessary loss of sales of the particular scalp care brand for the manufacturer.

Furthermore, retail shopping environments tend to be impersonal. The consumer is left basically alone to pick and choose the appropriate skin or hair care treatments that are most suited to his or her own needs and preferences. Even when a salesperson is available to assist the consumer with or make recommendations for treatment selections, the salesperson's assistance and recommendations are based upon a limited and/or subjective knowledge of the consumer and the consumer's treatment needs.

Methods of measuring moisture content have been developed in the past to determine the moisture level of skin or hair, and have relied on various techniques including resistance and capacitance measurements to obtain the desired indication.

### SUMMARY

In an aspect, the invention features a device for providing an indication of moisture present in skin of a user. The device includes a device body including a skin contact portion. A sensor circuit is located in the device body. The sensor circuit is configured to produce an output signal having a signal value that corresponds to moisture level in the skin with the skin contact portion in contact with the skin at a skin measurement location. A controller is in communication with the sensor circuit and is configured to determine the signal value of the output signal. The controller stores a first signal value in memory corresponding to a first output signal produced with the skin contact portion in contact with the skin of the user at a first skin measurement location. The controller stores a second signal value in memory corresponding to a second output signal generated with the contact portion in contact with the skin of the user at a second skin measurement location different from the first skin measurement location. The first and second signal values are used to indicate moisture present in the skin of the user at the second skin measurement location.

In another aspect, the invention features a device for providing an indication of moisture present in skin. The device includes a device body including a skin contact portion. A sensor circuit is located in the device body. The sensor circuit is configured to produce an output signal having a signal value that corresponds to moisture level in the skin with the skin contact portion in contact with the skin at a skin measurement location. A controller is in communication with the sensor circuit. Software is included that has controller-executable instructions implemented in a controller-readable medium defining logic which stores a first signal value in memory corresponding to a first output signal produced with the contact portion in contact with skin at a first skin measurement location and which stores a second signal value in memory corresponding to a second output signal generated with the contact portion in contact with the skin at a second skin measurement location different from the first skin measurement location. The first and second signal values are used to indicate moisture present in the skin at the second skin measurement location.

Implementations may include one of more of the following features. The second skin measurement location may be at the user's scalp. The first skin measurement location may be at the user's forehead.

In some embodiments, an electrode is located at the skin contact portion where the sensor circuit is used to measure impedance between the electrode in contact with the user's skin and the user's skin. The electrode may be moveable relative to the device body. In some implementations, the electrode is biased toward an extended position.

An interface module, such as a USB module, may be connected to the controller for use in communicating with a computer. In some embodiments, controller executable instructions include logic for calculating a difference between the second and first signal values.

The controller may store more than two signal values in memory, such as a third signal value in memory corresponding to a third output signal generated with the contact portion in contact with skin of a user. The signal may have a frequency and the device may include a pulse counter connected to the controller for use in determining the frequency of the signal where the pulse counter generates a pulse count that corresponds to the frequency. The controller may read the pulse count generated by the pulse counter to determine the signal value.

In some embodiments, a display such as an LED display may be connected to the controller. The controller may be located in the device body. In some embodiments, the controller stores the first and second signal values in memory located in the device body.

In another aspect, the invention features a skin treatment kit that includes a skin treatment product including a skin treatment composition and a skin moisture unit. The skin moisture unit is configured to provide an indication of moisture in a user's skin. The skin moisture unit and the skin treatment product are packaged together as a unit.

In some embodiments, the skin moisture unit and the skin treatment product are packaged together for retail sale. In certain implementations, multiple skin treatment products may be included where at least two of the skin treatment products have differing skin treatment compositions. The skin moisture measurement unit may be configured for use in selecting one or more skin treatment products from the multiple skin treatment products. The skin treatment product may be shampoo.

In one embodiment, the skin moisture measurement unit includes a device body including a skin contact portion. A sensor unit is located in the device body and is configured to produce an output signal having a signal value that corresponds to a moisture level in skin with the skin contact portion in contact with the skin at a skin measurement location. A controller is located in the device body and is in communication with the sensor circuit. The controller is configures to determine the signal value of the output signal.

In another aspect, the invention features a system for use in identifying a skin treatment. The system includes a skin moisture measurement unit having a skin contact portion. The skin moisture measurement unit includes a sensor circuit that is configured to produce an output signal having a signal value that corresponds to moisture level in the skin with the skin contact portion in contact with the skin at a skin measurement location. A controller is in communication with the sensor circuit. The controller is configured to determine the signal value of the output signal. A computer is configured to receive the signal value from the skin moisture measurement unit. The computer includes software that when executed by the computer is operable to map a calculated value determined using the signal value to a scale value that is used to indicate amount of moisture present in the skin.

Implementations may include one or more of the following features. The calculated value may be determined by calculating the difference between two signal values determined by the controller where each signal value is taken at a different skin measurement location using the skin moisture measurement unit.

In some embodiments, the skin moisture measurement unit includes an interface module for use in communicating with the computer. The skin moisture measurement unit may further include a display for displaying one or more of the signal value, the calculated value and the scale value.

In some implementations, multiple skin treatment products are included where each skin treatment product has a skin treatment composition. The skin moisture measurement unit may be configured for use in selecting one or more of the skin treatment products.

In another aspect, the invention features software for use in providing an indication of moisture present in skin. The software is embodied in computer-readable media and when executed is operable to map a reading value to a scale value that is used to indicate amount of moisture present in the skin. The reading value is calculated using two or more signal values stored in memory where the two or more signal values correspond to respective moisture readings taken at two or more differing skin measurement locations.

The details of one or more embodiments of the invention are set forth in the accompanying drawings and the description below. Other features, objects and advantages of the invention will be apparent from the description and drawings, and from the claims.

### BRIEF DESCRIPTION OF THE DRAWINGS

FIG. 1 is a diagrammatic illustration of a method and apparatus for indicating moisture content of skin;

FIG. 2 is a perspective, front view of multiple skin treatment products;

FIG. 3 is perspective, top view of an embodiment of a skin moisture measurement unit;

FIG. 3A is a detail, perspective view of an embodiment of an electrode assembly for use with the skin moisture measurement unit of FIG. 3;

FIG. 3B is a detail, side view of the electrode assembly of FIG. 3A in contact with a skin surface;

FIG. 4 is a circuit representation of an embodiment of a sensor circuit for use in the skin moisture measurement unit of FIG. 3;

FIG. 5 is a circuit representation of an embodiment of a counter circuit for use in the skin moisture measurement unit of FIG. 3;

FIG. 6 is a circuit representation of an embodiment of a circuit including a controller for use in the skin moisture measurement unit of FIG. 3;

FIG. 7 is a circuit representation of an embodiment of an interface circuit for use in the skin moisture measurement unit of FIG. 3;

FIG. 8 is a circuit representation of an embodiment of a beeper circuit for use in the skin moisture measurement unit of FIG. 3;

FIG. 9 shows the circuit representations of FIGS. 4-8 connected together;

FIG. 10 is a circuit representation of an embodiment of a circuit including a preamplifier and a buffer for use in the skin moisture unit of FIG. 3;

FIG. 11 illustrates a method of using the skin moisture measurement unit of FIG. 3; and

FIG. 12 is a perspective view of an embodiment of a skin treatment kit.

### DETAILED DESCRIPTION

For purposes of describing various embodiments of the invention, the description below will focus on determining moisture content at the scalp of a human user. However, these embodiments are exemplary as aspects may apply to moisture measurements at other skin locations. Additionally, embodiments of the invention may be used to indicate moisture content of other mammals, such as certain pets.

All documents cited in the Detailed Description are incorporated herein by reference. The citation of any document is not to be construed as an admission that it is prior art with respect to the present invention.

A. Introduction

As used herein, the term “skin” refers to the membranous tissue forming the external covering of the human body including, for example, the external covering of the face, neck, chest, back, arms, hands, legs and scalp.

The term “scalp” refers to the skin covering the top of the head and the term “forehead” refers to the part of the face between the eyes and the scalp.

As used herein, “moisture content” refers to the amount of water present in the skin.

Determination of moisture content in skin is used to quantify various physical and cosmetic characteristics of the skin. For example, a scalp with a low moisture content may be indicative of an unhealthy scalp and may exhibit signs of dandruff, including flakes, dryness, tightness, itchiness, redness and/or irritation. Skin hydration is a function of its normal biological activity that results in a continuous moisture flux from within the body to the environment. Improvement in the skin barrier function results in greater skin hydration and less moisture loss. The physical and cosmetic characteristics of the skin may be improved, for example, with treatments that restore skin normal conditions and improve the skin's barrier function. Improvement of barrier function, in turn, also results in protecting the skin from environmental, physical, chemical, or biological insults and results in an overall improvement in skin health. Treatments having a formulation for increasing skin moisture content may include certain hair care products, personal care products, and beauty care products, including, but not limited to, lotions, creams, gels, tonics, after shave, sticks, sprays, ointments, pastes, powders, mousse, shampoos, conditioners, oils, colorants, and biomedical and dermatological treatments.

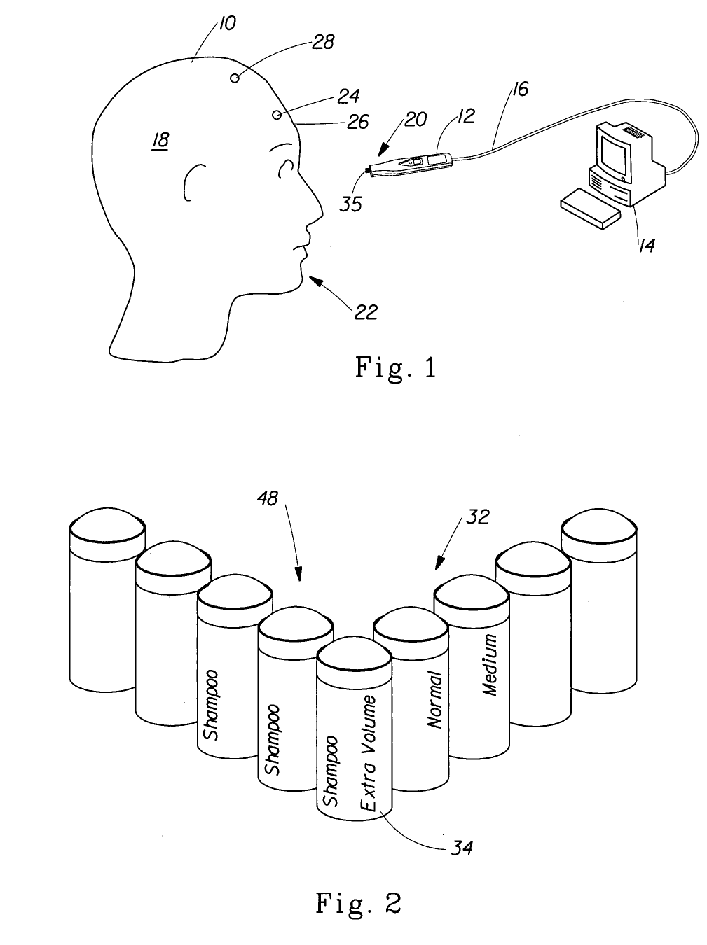

Referring to FIG. 1, a method and apparatus for indicating moisture content in a region of the scalp 10 includes a moisture sensor measurement unit (MSMU) 12 that is capable of communicating with a computer 14 via a connection 16. In some embodiments, connection 16 is a wired connection, such as through use of a USB connection, serial port or S cable connection, etc. In other embodiments, connection is a wireless connection, such as via a wireless RF connection, infra-red connection. In an alternative embodiment, MSMU 12 may not communicate with computer 14 and, in some instances, be configured for use independent of the computer.

The MSMU 12 obtains a skin moisture value of a user's skin 18 at a skin measurement location. In FIG. 1, by contacting a contact portion 20 of the MSMU 12 to skin 18 of the user 22 at a first skin measurement location 24, the MSMU can obtain a moisture value that is related to moisture content at the first skin measurement location at the user's forehead 26. The MSMU 12 may include an electrode 35 (e.g., one or more electrodes, such as two, three, four or more electrodes) at the contact portion 20 for contacting the skin. This first moisture value associated with moisture content at the first skin measurement location 24 can be used as a datum value to which other skin moisture values taken at differing skin measurement locations can be compared. For example, MSMU 12 may obtain a second skin moisture value by contacting contact portion 20 to the user's skin 18 at a second skin measurement location 28 at the user's scalp 10. The second skin moisture value can be compared to the first skin moisture value, for example, to indicate moisture content of the scalp 10. While two skin measurement locations 24 and 28 are shown, skin moisture values can be obtained at more than two skin measurement locations, such as three skin measurement locations or more.

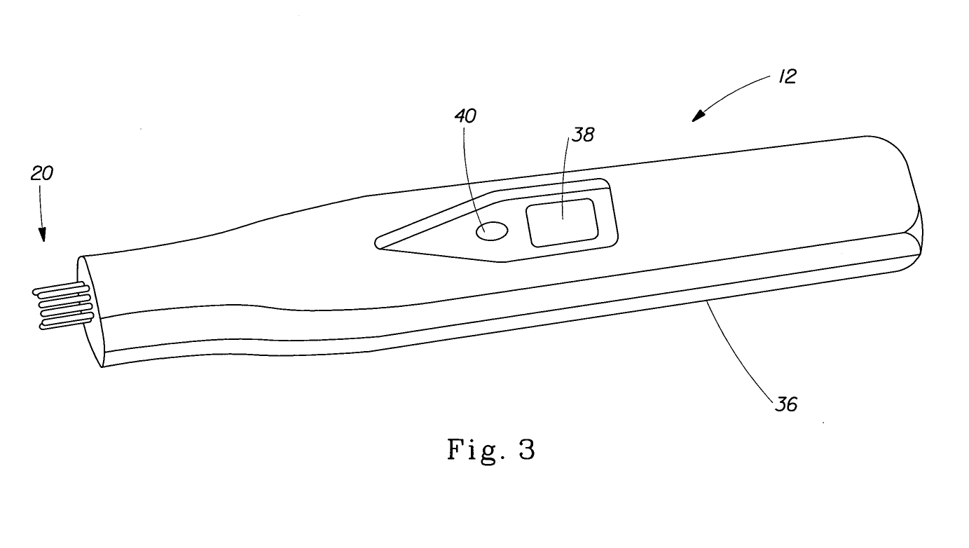

Skin moisture values obtained by the MSMU 12 can be sent to and/or retrieved by computer 14. Computer 14 may include logic for use in processing the skin moisture values and presenting the processed data, for example, to the user 22 or to someone else assisting the user in a meaningful format. In one embodiment, computer 14 uses a comparison between the first and second skin moisture values to determine a recommended product from a set of products 32. For example, referring also to FIG. 2, a comparison between the first and second skin moisture values may indicate that the second skin measurement location 28 at the scalp 10 has a substantially lower moisture content than the moisture content at the first skin measurement location 24 at the forehead 26. In this instance, the computer 14 may recommend a shampoo product 34 from a set of shampoo products 43 that is formulated particularly to increase moisture content of relatively dry scalps.

B. Exemplary Moisture Sensor Measurement Unit (MSMU)

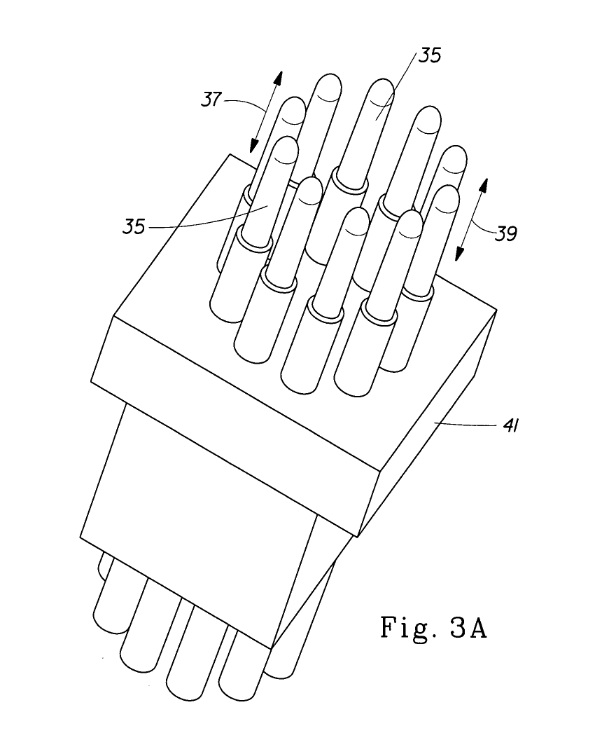

Referring to FIG. 3, MSMU 12 includes a body 36 having contact portion 20. Body 36 can be shaped and sized to fit comfortably within a user's hand. A display 38 (e.g., an LED display) is viewable by the user and is used to display, for example, the skin moisture values obtained by the MSMU 12. Other information may be displayed by the display 38. Power to the MSMU 12 can be manually turned off using interface component 40, here, shown as an ON/OFF button. In some implementations, other component 40 types may be used such as a sliding switch, toggle switch, and/or in some embodiments, the computer 14 may be used to discontinue power to the MSMU 12.

As will be described in detail below, MSMU 12 utilizes changes in skin permittivity and skin resistivity at differing locations of a user's skin to indicate relative moisture content at one or more of the skin locations. A relatively high moisture content of skin at a skin location reduces complex skin impedance of the skin and decreases the resistance and capacitance of a sensor circuit in contact with the skin at the skin location. A relatively low moisture content of skin increases the complex skin impedance of the skin and increases the resistance and capacitance of the sensor circuit.

Referring to FIG. 3A, the electrodes 35 are located at the contacting portion 20 of the MSMU 12. The electrodes 35 are connected to the sensor circuit and used to bring the sensor circuit in contact with the skin. As illustrated by arrows 37 and 39, at least one or more, such as all of the electrodes 35 are moveable, in the illustrated embodiment, linearly relative to housing 41 (and body 36 ). In some embodiments, the electrodes 35 are biased toward a fully extended position, e.g., by a spring (not shown).

Referring also to FIG. 3B, by providing moveable electrodes 35 that are biased toward their respective fully extended positions, uniform contact can be achieved between the skin and the MSMU 12 regardless of topology of skin 18 surface contour. Additionally, the biased, moveable electrodes 35 can allow for more reproducible skin moisture measurements by reducing the impact of differing applied pressures, for example, between different users and/or different readings bringing the MSMU 12 in contact with the skin 18.

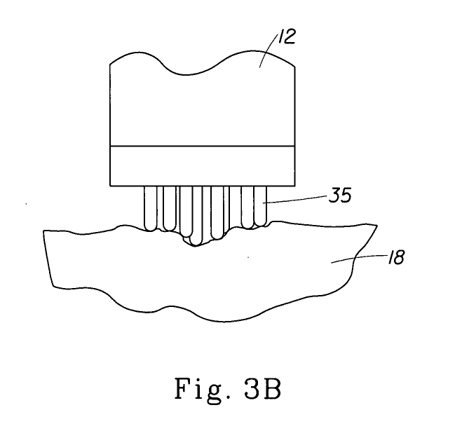

Disposed in the body 36 of the MSMU 12 are electronics for use in providing an indication of moisture content. Referring to FIG. 4, sensor circuit 42 is used to generate a signal having a frequency that corresponds to moisture content in skin at the skin measurement location. The sensor circuit 42 utilizes the RC component present in the skin by contacting the contact portion 20 of the MSMU 12 to the skin, which can alter the frequency of the signal using the permittivity and resistivity changes in the skin as noted above. A change in the moisture content of the skin reflects as a change in impedance of the sensor circuit 42.

In operation, sensor circuit 42 includes an integrated timer 44 and generates a signal having a predetermined open frequency (e.g., of about 2.4 MHz) with the skin contact portion 20 removed from the skin. Sensor circuit 42 includes a variable resistor 30 (e.g., a 3.3 Kilo Ohm variable resistor) as a trimmer for use in maintaining the predetermined open frequency. When the contact portion 20 of the MSMU 12 is brought into contact with skin, this contact causes a change in the impedance of the sensor circuit 42, which changes the frequency of the signal. Sensor circuit 42 may be guarded against electrostatic discharge (ESD), for example, using a bridge network of diodes 46 located between ground and VDD supply.

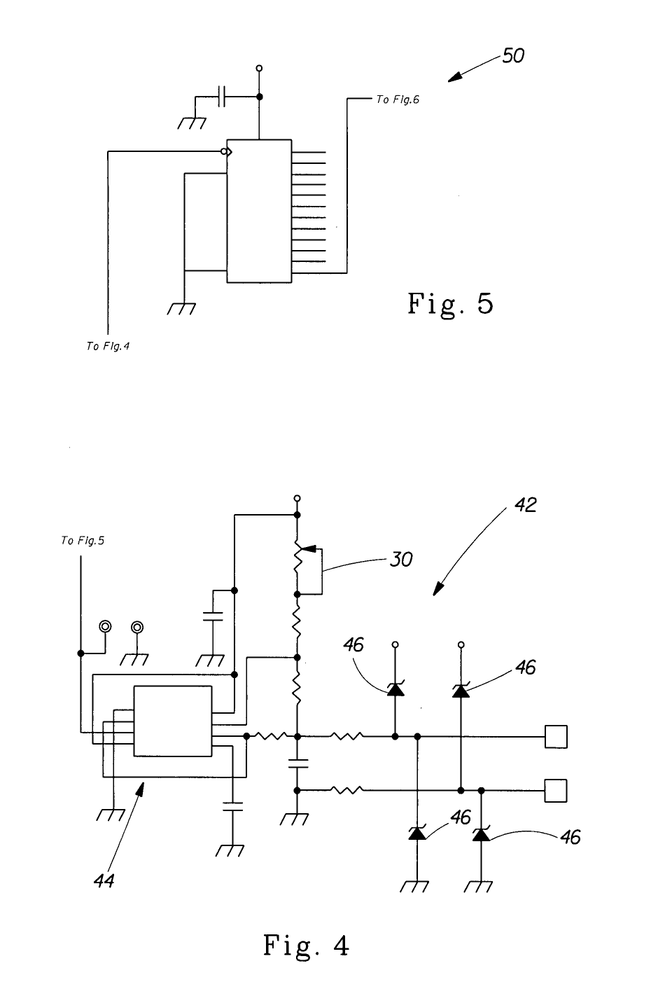

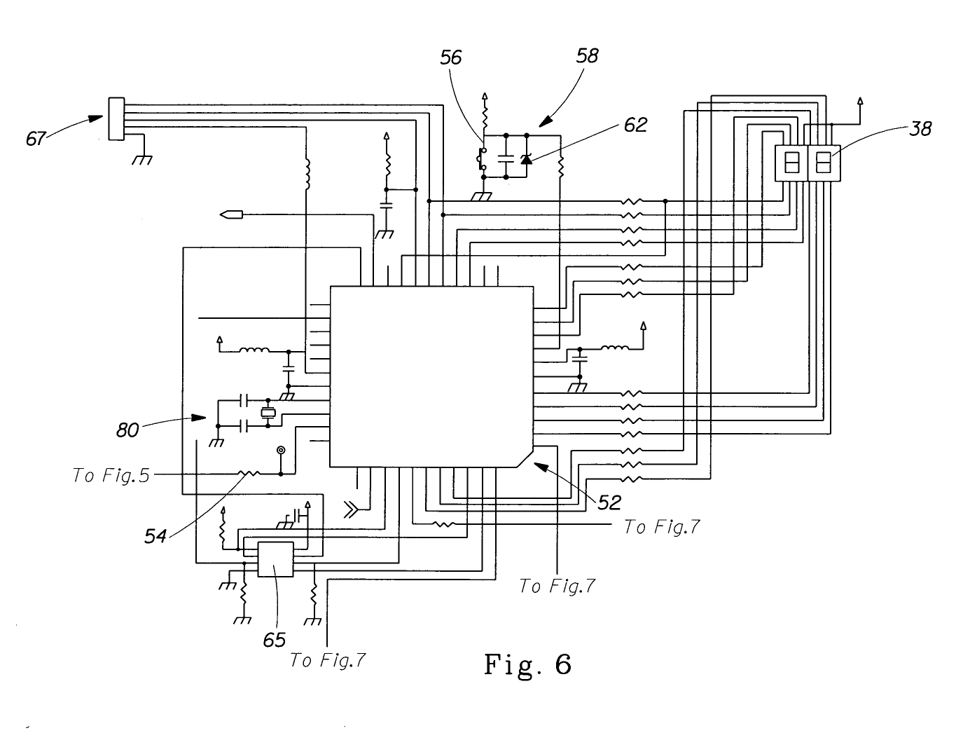

Referring to FIG. 5, a binary counter 50 (e.g., a 14 bit CMOS binary counter in a free mode configuration without utilizing a reset option) is connected to the sensor circuit 42 between the sensor circuit and a microcontroller 52 (FIG. 6). The binary counter 50 is used as a pre-scaler that augments the signal for microcontroller input. In this example, binary counter 50 is connected to the microcontroller 52 only at pin 14 through a resistor 54 (e.g., a 10 k resistor; see FIG. 6). However, other configurations are possible.

Referring to FIG. 6, microcontroller 52 (e.g., a PIC microcontroller 16F877A) acts as a central processing unit for the MSMU 12 by controlling and communicating with the peripheral circuits. Microcontroller 52 is used to measure the incoming pulse count from the binary counter 50, for example, upon actuation of switch 56 such as a momentary switch forming interface component 40. A switch depressed detector 58 can incorporate a guard circuit 60 for ESD protection using a diode 62. The pulse count can be stored in memory 65 (e.g., an electrically-erasable programmable read-only memory (EEPROM)) as a numerical skin moisture value, for example, in Hz. In some embodiments, the skin moisture value is displayed on display 38, for example, as a two-digit numerical value on the two-digit LED display 38. In some embodiments, there is a provision made in the microcontroller 52 that the microcontroller 52 can be programmed using a circuit serial programming component 67 while the microcontroller 52 is placed on the circuit board (not shown), which may provide flexibility in reprogramming. In certain embodiments, the microcontroller 52 operates at about 4 MHz using a crystal oscillator 80.

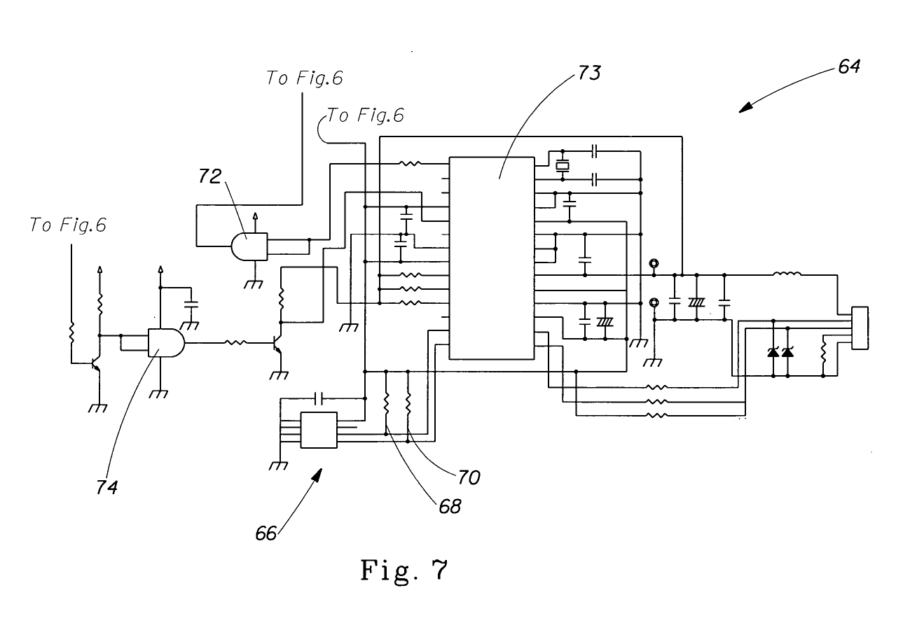

Referring now to FIG. 7, microcontroller 52 may be connected to computer 14, for example, via an interface module 64 that can convert an output signal from the microcontroller 52 to an input compatible for computer 14. In an embodiment, interface module 64 is a USB interface module that uses an IC PL-2303 USB to serial bridge controller 73 to convert the output signal from the microcontroller 52 (e.g., in RS 232 protocol) into a USB protocol. In some embodiments, both the microcontroller 52 and the PL-2303 USB to serial bridge controller 73 communicate in serial communication protocol, for example, at 4800 kbs baud rate. The PL-2303 serial to bridge controller 73 may include an external memory 66 such as EEPROM connected thereto, for example, using I²C logic. Pull-up resistors 68 and 70 are included in clock and data signal lines to improve data transmission speed and reliability. Input pins (not shown) that are connected to the USB bus signal of the computer 14 are guarded against ESD. The signal line (RX) to send data from the microcontroller 52 to the computer 14 and the signal line (TX) to send data to the microcontroller 52 from the computer 14 are fed through a buffer stage that includes AND gates 72 and 74.

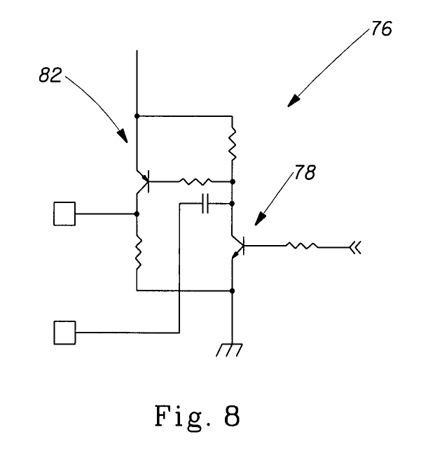

Referring to FIG. 8, circuit 76 is used to provide a user with an indication of a satisfactory reading using the MSMU 12. A preamplifier 78 and buffer 82 are connected back-to-back to form the circuit 76. A beeper algorithm resides in memory of the microcontroller 52 that is used to generate a square wave, which, in the illustrated embodiment, is in an audible frequency range. The square wave signal is sent to preamplifier 78 (e.g., including a bipolar junction transistor) to drive a beeper speaker and buffer 82 (e.g., including a PNP transistor) provide signal isolation.

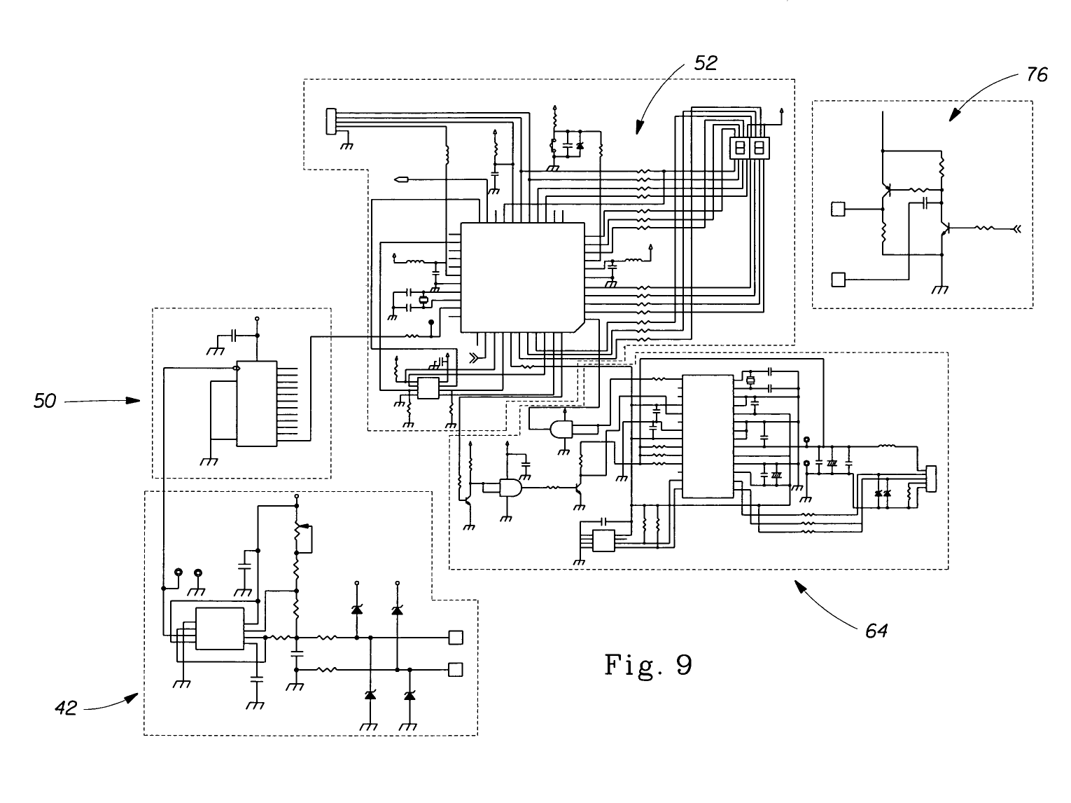

FIG. 9 shows connections between sensor circuit 42, binary counter 50, interface module 64, indicator circuit 76 and/or microcontroller 52.

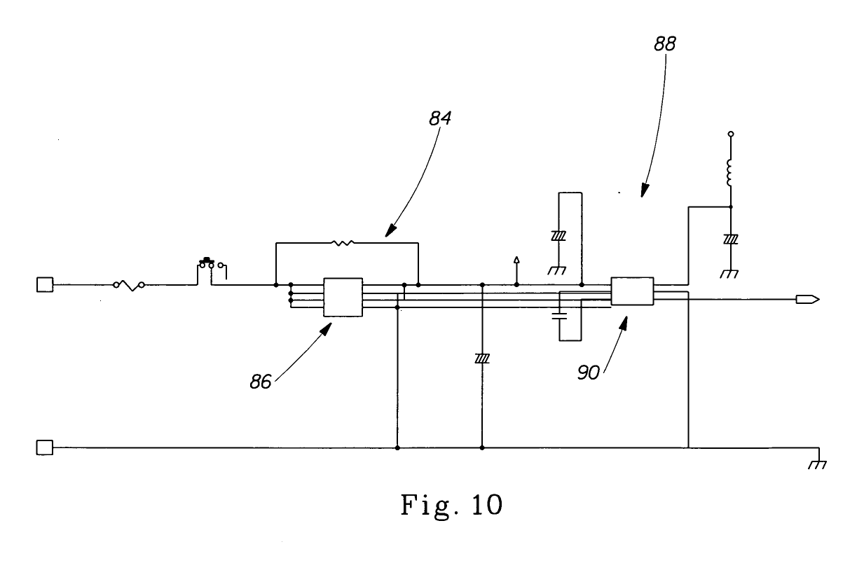

Referring now to FIG. 10, a polarity detection circuit 84 is used for polarity protection. In some embodiments, the polarity protection circuit 84 includes a transistor 86 (e.g., a TPC 8305) that can include, for example, two field-effect transistors (e.g., metal-oxide-semiconductor field-effect transistors (MOSFETs)). In some embodiments, mechanical polarity protection may be incorporated in the MSMU 12.

In some implementations, most of the peripheral circuits such as the sensor circuit 42, the binary counter 50 and the indicator circuit 76 need not be in operation after the skin measurement value has been recorded. In some embodiments, a provision may be made to separate the power supply connection for the peripheral circuits 42, 50, 76 in that the peripheral circuits 42, 50 and 76 may be disconnected from the power supply with the other circuits remaining connected to the power supply. To this end, a charge pump circuit 88 can be used to generate a power supply (e.g., a 3.3 V power supply) from a battery source (e.g., a 3 V battery source) based on a command from the microcontroller 52. Microcontroller 52 is directly connected to a battery source and, in some embodiments, can operate for as long as it is connected thereto. Whenever the microcontroller 52 is ready to initiate a measurement cycle, the microcontroller may send a signal to a charge pump chip 90 to activate the peripheral circuits 42, 50 and 76. Once the measurement cycle is completed, the microcontroller 52 may send a signal to the charge pump chip 90 to disconnect the power supply to the peripheral circuits 42, 50 and 76.

C. Methods and Logic for MSMU Usage

As noted above with reference to FIG. 1, MSMU 12 may be connected to a computer 14. In these embodiments, the computer 14 may receive data such as the skin moisture values from the MSMU, for example, via the USB connection and process the data further. The computer 14 may also include logic for directing the user through steps of MSMU use and/or provide viewable reports that present information to the user.

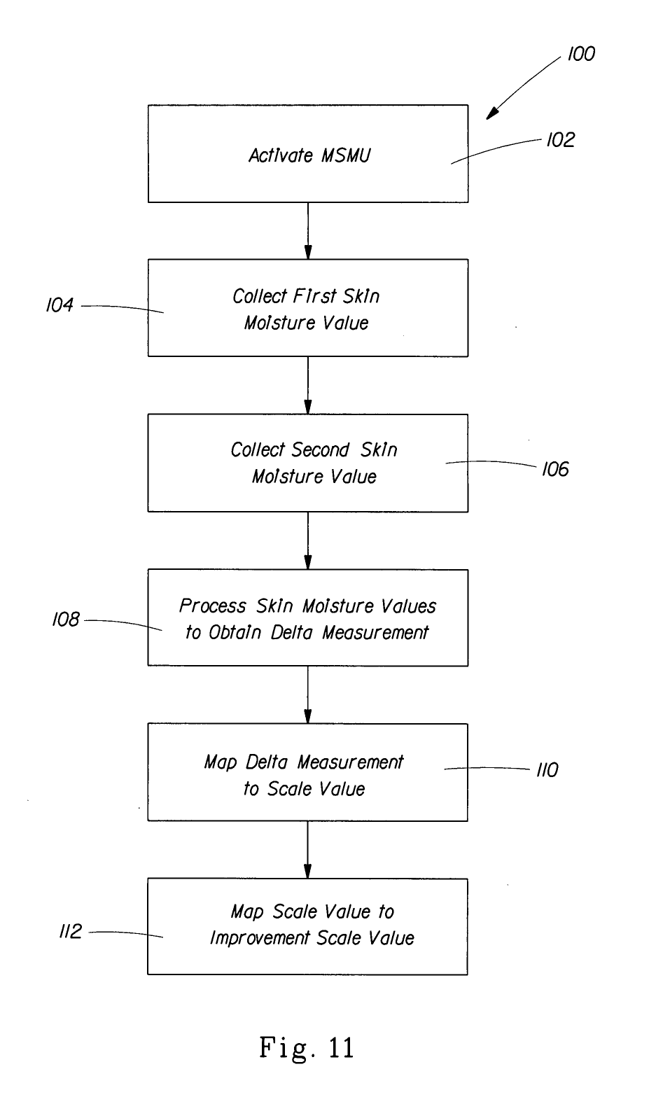

FIG. 11 shows a method 100 of MSMU 12 use that includes activating 102 the MSMU, for example, by pressing the ON/OFF button 30 (FIG. 3). At step 104, the contact portion 20 of the MSMU 12 is contacted to a user's skin at a first skin measurement location, for example, at the forehead and a first skin moisture value m1 is collected. In some embodiments, an audible beep or other sound indicates a successful reading. At step 106, the contact portion 20 of the MSMU 12 is contacted to a user's skin at a second skin measurement location, for example, at the user's scalp and a second skin moisture value m2 is collected. Another audible beep or other sound may be used to indicate a second, successful reading. More than two skin moisture values may be collected, such as three skin moisture values. At a processing step 108, the skin moisture values are processed.

A Delta Measurement (e.g., m2−m1) may be performed by the MSMU 12 at step 108, for example, using the microcontroller 52 and logic and m1 and m2 saved in memory accessible to the microcontroller. In some embodiments, each skin moisture value m1 and m2 may be sent to the computer 14 for processing the skin moisture values including the Delta Measurement. In yet another embodiment, the Delta Measurement may be calculated by a user, for example, manually or through use of a calculator. In these embodiments, the MSMU 12 (and/or computer 14) may display values for m1 and m2 to allow the user to perform the calculations.

Referring still to FIG. 11, at mapping step 110 the Delta Measurements are mapped to a scale value. Table I below is an exemplary table for mapping the Delta Measurements to a Scale Value.

TABLE I

| Delta Measurement | | |
|---|---|---|
| (MHz) | Scale Value | Scalp Condition |
| >=0.9 | 5 | Dry |
| 0.79-0.89 | 10 | Dry |
| 0.68-0.78 | 20 | Dry |
| 0.54-0.67 | 30 | Dry |
| 0.43-0.53 | 40 | Dry |
| 0.34-0.42 | 50 | Dry |
| 0.25-0.33 | 60 | Moderate |
| 0.15-0.24 | 70 | Moderate |
| 0.06-0.14 | 80 | Moderate |
| 0.04-0.05 | 90 | Moderate |
| <=0.03 | 95 | Healthy |

In some embodiments, the Scale Value is displayed to the user by, for example, the MSMU 12 using display 38 and/or by the computer 14. In one embodiment, a Scalp Condition associated with each Scale Value is displayed. The above mapping step 110 may be performed by the MSMU 12 (e.g., using microcontroller 52), by the computer 14 (e.g., using a processor remote from the MSMU) or manually. In some embodiments, a suitable mapping table and mapping logic may be supplied by a software package to the computer 14. In some implementations, a mapping table, such as Table I above may be provided to the user or to another assisting the user.

In a show improvement mapping step 112, the Scale Value may be mapped to an Improvement Scale Value. The Improvement Scale Value may be indicative of potential skin moisture improvement that can be achieved, for example, should the user use a particular product or series of products. Table II illustrates an Improvement Scale Value mapping table example.

TABLE II

| Scale Value | Improvement Scale Value |
|---|---|
| 5 | 50 |
| 10 | 50 |
| 20 | 50 |
| 30 | 50 |
| 40 | 50 |
| 50 | 90 |
| 60 | 90 |
| 70 | 90 |
| 80 | 90 |
| 90 | 95 |
| 95 | 95 |

In some embodiments, the Scale Value is mapped to the Improvement Scale Value upon user command using a user interface, for example, at the computer 14 and/or at the MSMU 12. For example, a user may press a switch of the MSMU 12 or strike a key of the computer 14 keyboard to display the Improvement Scale Value after the Scale Value has been displayed. In some implementations, the Scale Value may be mapped to the Improvement Scale Value automatically. The Improvement Scale Value can be displayed by the MSMU 12 and/or computer 14 and can give the user an indication whether and in some cases to what degree scalp moisture content can be improved.

Based at least in part on the measurements and values calculated, a product may be recommended, for example, by the computer 14 and/or by a person, for example, in a store. Various methods of using MSMU 12 are described in detail in pending U.S. patent application entitled “Methods for Measuring Moisture Content of Skin” (Docket No. 10118P) and in pending U.S. patent application, entitled “Methods for Retail Measurement of Skin Moisture Content” (Docket No. 10119P) both applications being filed on the same day as the instant application.

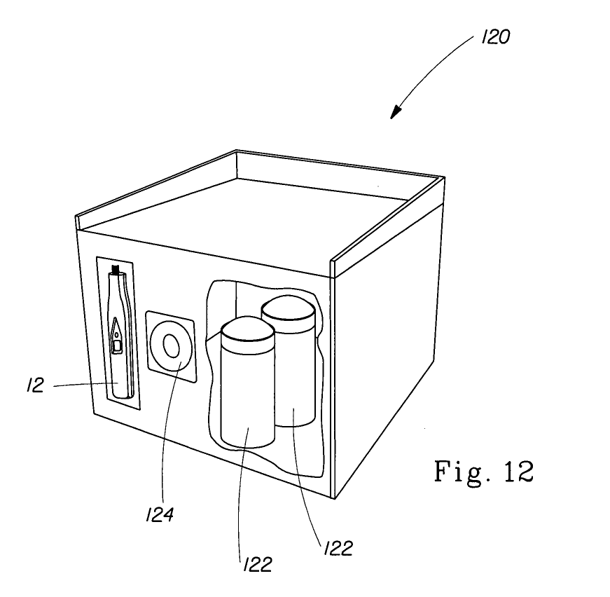

Referring now to FIG. 12, a kit 120 includes the MSMU 12 and one or more product 122 for use in improving moisture content of skin. Kit 120 may be available for retail sale and purchase by a customer and/or may be packaged for use by someone other than the customer to assist the customer during product selection (e.g., a dermatologist, a store employee, a supplier employee, a salon employee, etc.). In some embodiments, the kit 120 includes software 124 for use by a computer 14 for processing the skin moisture values and/or mapping, such as those described above. Kit 120 may include other items, such as batteries, cable (e.g., USB cable), printed mapping tables such as those described above, and the like.

D. Example

The following example is given solely for the purpose of illustration and is not meant to be construed as a limitation as many variations are possible.

To determine a correlation between moisture content of the scalp and dandruff, the scalp moisture content of consumers was measured using MSMU 12. The scalp moisture content for each consumer was determined as follows: One reading was taken from the forehead using the MSMU 12 and two readings were taken from the scalp using the MSMU. The readings were converted into a Delta Measurement using the MSMU 12. In addition, each consumer was assigned an Adherent Scalp Flaking Score (ASFS). The ASFS for each consumer was determined by having a qualified grader examine an octant of the consumer's scalp and then assigning a flake grade to that octant. Exemplary results are shown below by Table III.

TABLE III

| | Delta | ASFS | |
|---|---|---|---|
| Number of | Measurement | Octant | |
| Consumers | (KHz) | Grade | Scalp Condition |
| 61 | 395.164 | 8-10 | Severe Flaking |
| 235 | 308.766 | 4-6 | Moderate Flaking |
| 169 | 230.527 | 0-2 | No Flaking |

A number of detailed embodiments have been described. Nevertheless, it will be understood that various modifications may be made. For example, the MSMU 12 may be usable without a computer (i.e., the MSMU 12 may be a stand-alone device). In these embodiments, the MSMU 12 may include logic for performing some or all of the steps described above without the use of a computer 14. In some instances, the MSMU 12 may not be connected to a computer 14, but a computer 14 may be used to perform certain desired functions such as mapping, for example, through manual entry of relevant data into the computer. In some embodiments, the MSMU 12 has a Sleep Mode (e.g., if the MSMU 12 is idle for about 60 seconds or more) that can be used to conserve battery power. In some embodiments, the microcontroller 52 may check a battery power level. If the battery power level is below a predetermined value, the MSMU 12 may sound one or more Beeps and/or display 38 may provide a visual indication of low battery power, e.g., by displaying “LO.” In some cases, if a battery with low power is detected, the MSMU 12 may not allow any additional measurements to be taken until the battery is replaced. In certain embodiments, data from memory of the MSMU 12 can be downloaded to the computer 14, for example, via the USB connection and upon download the memory of the MSMU may be cleared. Memory of the MSMU 12 may be checked each time or certain times that the MSMU is activated. If the memory of the MSMU 12 is full, an indication of this (e.g., “FL”) can be displayed using display 38 for a period of time such as five seconds and then the memory can be cleared. Accordingly, other embodiments are contemplated.

What is claimed is:

1. A device for providing an indication of moisture present in skin of a user, the device comprising:
a device body including a skin contact portion comprising at least one moveable electrode and
a sensor circuit in the device body, the sensor circuit configured to produce an output signal having a signal value that corresponds to moisture level in skin when the skin contact portion of said device body is in contact with skin at a skin measurement location.

2. The device of claim 1, wherein said moveable electrode is moveable by means of a spring.

3. The device of claim 2, wherein said moveable electrode is amongst a plurality of moveable electrodes.

4. The device of claim 1, wherein said moveable electrode is amongst a plurality of moveable and stationary electrodes.

5. The device of claim 3, wherein at least one moveable electrode is biased toward an extended position.

6. The device of claim 1, wherein the skin measurement location is at the user's scalp.

7. The device of claim 1, wherein the skin measurement location is at the user's forehead.

8. The device of claim 1, wherein at least one moveable electrode provides measurement for impedance between the electrode in contact with the user's skin and the user's skin.

9. The device of claim 1, wherein the skin contact portion is connected indirectly to said device body.
